(19) United States
(12) Patent Application Publication
(10) Pub. No.: US 2025/0032412 A1
(43) Pub. Date: Jan. 30, 2025

(54) SELF-EMULSIFYING OIL-IN-WATER MICROEMULSION OR NANOEMULSION, AND EMULSIFYING COMPOSITION

(71) Applicant: URSAPHARM ARZNEIMITTEL GMBH, Saarbrücken (DE)

(72) Inventors: Ute STEINFELD, St. Ingbert (DE); Hyeck HEE LEE, St. Ingbert (DE); Frank HOLZER, St. Ingbert (DE); Markus MAHLER, Völklingen (DE)

(73) Assignee: URSAPHARM ARZNEIMITTEL GMBH, Saarbrücken (DE)

(21) Appl. No.: 18/708,995

(22) PCT Filed: Nov. 11, 2022
(86) PCT No.: PCT/EP2022/081645

§ 371 Date: May 9, 2024

(30) Foreign Application Priority Data
Nov. 11, 2021 (DE) 10 2021 212 692.8

(51) Int. Cl. A61K 9/107; A61K 8/02; A61K 8/06; A61K 8/92; A61K 31/155; A61K 31/167; A61K 31/192; A61K 31/196; A61K 31/55; A61K 31/555; A61K 31/573; A61K 31/60; A61K 31/616; A61K 31/65; A61K 45/06; A61K 47/10; A61K 47/12; A61K 47/22; A61K 47/26; A61K 47/36; A61K 47/38; A61Q 19/00

(52) U.S. Cl. CPC A61K 9/1075; A61K 8/0291; A61K 8/062; A61K 8/068; A61K 8/922; A61K 31/155; A61K 31/167; A61K 31/192; A61K 31/196; A61K 31/55; A61K 31/555; A61K 31/573; A61K 31/60; A61K 31/616; A61K 31/65; A61K 45/06; A61K 47/10; A61K 47/12; A61K 47/22; A61K 47/26; A61K 47/36; A61K 47/38; A61Q 19/00; A61K 2800/10

(57) ABSTRACT

The present invention relates to a self-emulsifying oil-in-water microemulsion or nanoemulsion, containing or consisting of at least one surface-active antioxidant, at least one zwitterionic substance, and at least one active substance. The active substance can be effectively emulsified even in the case of low water solubility, and therefore its bioavailability is significantly increased. In many cases, the improved bioavailability means that the active substance concentration can be accordingly reduced and the biocompatibility can be increased as a result. The present invention also relates to an emulsifying composition, by means of which active substances can be effectively emulsified.

The present invention relates to a self-emulsifying oil-in-water microemulsion or nanoemulsion, containing or consisting of at least one surface-active antioxidant, at least one zwitterionic substance, and at least one active substance. In this case, the active substance can be effectively emulsified even with low solubility and therefore its bioavailability is considerably increased. Accordingly, by means of the improved bioavailability, the active substance concentration can frequently be reduced and therefore the biocompatibility can be increased. The present invention also relates to an emulsifying composition by means of which active substances can be effectively emulsified.

Chemical changes to active substances and their formulations take place by hydrolysis, redox processes, photochemical radical reactions, and the like. As a result, the shelf life of cosmetics, medical products, and pharmaceuticals is reduced.

US 2013/0108674 A1 relates to ophthalmic pharmaceutical compositions in the form of a microemulsion which can carry liposoluble or slightly water-soluble active substances and contain an emulsifying agent consisting of d-a-tocopheryl polyethylene glycol 1000 succinate (TPGS), an oily component consisting of medium-chain triglycerides (MCT), and an ophthalmologically acceptable aqueous phase. The invention also relates to ophthalmic compositions for use as a tear substitute, which are formulated similarly to the previous compositions but do not contain a pharmaceutical active substance and contain, in the aqueous phase, polysaccharide polymers or cellulose derivatives, which are known as components for artificial tears. The described preparations are self-emulsifying systems (SMEDDS) which are characterized by high stability and by a particularly low concentration of surfactants, and are suitable for topical ocular administration.

EP 1464 341 A1 discloses a formulation for ophthalmic use in the form of an aqueous solution which contains ubiquinone Q10 in association with vitamin E TPGS. The latter is an effective antioxidant which not only acts synergistically with ubiquinone, but also behaves as an effective solubilizer for the ubiquinone itself, which in its absence would be totally insoluble in an aqueous environment.

With pharmaceutical active substances which are insoluble in water, are only sparingly soluble in water, or are poorly soluble in water, such as bibrocathol, an as yet unsolved problem is to provide these substances as stable aqueous formulations, using which the problem of metal ions, such as bismuth, complexing from metal-ion-containing active substances during storage is simultaneously solved.

Bibrocathol is a bismuth-containing drug substance from the group of antiseptic agents. It is generally slightly soluble in hydrophilic and lipophilic substances, and is currently marketed in the form of a suspension-based eye ointment, e.g. for the treatment of eyelid margin inflammation, seborrhea, or staphylococcal accumulations in the eye area.

The problem addressed by the present invention is therefore to provide a self-emulsifying composition by means of which not only active substances that are slightly soluble in water can be emulsified, but which also provides equally excellent protection of the emulsified substances, for example against hydrolysis and/or oxidation and/or complexing of metal ions that are contained. This problem is solved by means of a self-emulsifying oil-in-water microemulsion or nanoemulsion according to the features of claim 1 in relation to an emulsifying composition having the features of claim 21. The respectively dependent claims amount to advantageous developments here.

The present invention thus relates to a self-emulsifying oil-in-water microemulsion or nanoemulsion, containing or consisting of
at least one surface-active antioxidant,
at least one zwitterionic substance, and
at least one active substance that is at least sparingly soluble in water, wherein the at least one active substance that is at least slightly soluble in water is present in micelles so as to be encapsulated at least in part by the at least one surface-active antioxidant.

The term “at least sparingly soluble in water” is used here in an identical manner to the definition in the European Pharmacopoeia. With regard to solubility, the European Pharmacopoeia, 8th Edition, Volume 2, page 5614 ff. (1.4 Monographs) defines the solubility of substances at a temperature of 15° C. to 25° C. as follows:

| | V(solvent) in ml per g | |
|---|---|---|
| Descriptive term | of substance | g · l⁻¹ solvent |
| very soluble | <1 | >1000 |
| freely soluble | 1 to 10 | 100 to 1000 |
| soluble | 10 to 30 | 33 to 100 |
| sparingly soluble | 30 to 100 | 10 to 33 |
| slightly soluble | 100 to 1000 | 1 to 10 |
| very slightly soluble | 1000 to 10000 | 0.1 to 1 |
| practically (almost) insoluble | >10000 | <0.1 |

The term “at least sparingly soluble in water” is thus understood in the present invention such that the at least one active substance, i.e., in the case of a single active substance, the active substance, or, in the case of a plurality of active substances, each individual active substance, has a solubility in water at a temperature of 15° C. to 25° C. that is such that a minimum of 30 ml water needs to be used to completely dissolve a gram of the at least one active substance.

It could surprisingly be found that, using the microemulsion or nanoemulsion according to the invention, active substances that are at least sparingly soluble, slightly soluble, very slightly soluble, or almost insoluble in water can also be effectively emulsified under the formation of micelles. Owing to the use of surface-active antioxidants instead of conventional emulsifiers in combination with one or more zwitterions, the O/W microemulsions or nanoemulsions according to the invention also surprisingly provide effective protection, for example against hydrolysis and/or oxidation of the active substances and also the entire formulation.

According to the invention, it is thus possible to formulate sparingly water-soluble active substances as stable oil-in-water (O/W) microemulsions or nanoemulsions. In this case, the active substance can preferably be embedded in a lipophilic carrier substance in micelles in a protective manner. For this purpose, a surface-active antioxidant is selected in order, on one hand, to formulate and stabilize the slightly soluble active substance in micelles and, on the other hand, to protect the formulation against degradation processes during storage and to improve the shelf life of the formulation.

Accordingly, cosmetic, medical-product-based, and pharmaceutical products can be effectively protected against degradation, in particular hydrolysis and/or oxidative degradation.

According to a specific embodiment, it is provided that one part of the active substance that is at least sparingly soluble in water, which e.g. can be a metal-containing active substance, is present in solution and one part is encapsulated in micelles. For slightly soluble active substances, it is conceivable that the active substance is finely dispersed in the solution, i.e. as a suspension which additionally contains the active substance encapsulated in micelles.

Owing to the fact that one part of the active substance that is at least sparingly soluble in water, which e.g. can be a metal-containing active substance, is present in solution and one part is encapsulated in micelles, this thus results in an immediate effect by means of the directly available active substance in the solution and a retard effect by means of the active substance released from the micelles over time. This is particularly advantageous in the formulation of the self-emulsifying oil-in-water microemulsion or nanoemulsion according to the invention as eye drops.

This combination of an immediate effect and a retard effect ensures rapid availability and a sustained, longer-lasting effect of the active substance. Muco-adhesive viscosity improvers having long-lasting adhesion to the surface of the eye, such as xanthan gum, HPMC, etc., encourage this mechanism of action.

The microemulsion or nanoemulsion according to the invention is suitable in particular as an ophthalmic formulation, e.g. as eye drops and/or suspension-based eye drops. In comparison with a previously known suspension-based ointment, which provides the hitherto only option of applying ophthalmically active substances having low water solubility (in particular bibrocathol) to or in the eye, this provides the following advantages:
no visual impairment (blurred vision)
more rapid distribution, availability, and action of the active substance in the aqueous formulation on the surface of the eye
application in smaller volumes is possible
potentially less of a foreign body sensation, since the drops correspond to the tear film in consistency and are less viscous than an ointment.

This also applies to suspension-based eye drops, since these have an aqueous base and not an ointment base.

It has surprisingly been found that a stable formulation of active substances that are at least sparingly soluble in water and/or are sensitive to hydrolysis is possible in the oil-in-water emulsion according to the invention based on surface-active antioxidants and a zwitterionic substance, in particular a zwitterionic buffer. In this case, the surface-active antioxidants have a kind of “double function”, i.e. they simultaneously allow for effective emulsification of the active substance and for protection against degradation, in particular oxidation and/or hydrolysis. Here, the active substances are in particular selected from lipophilic active substances.

The zwitterionic substance, e.g. the zwitterionic buffer, serves to stabilize the active substance that is at least sparingly soluble in water, e.g. the metal-ion-containing active substance in the solution, such that no sedimentation or phase separation can be observed. This is based on the stabilizing effect of the buffer zwitterions by means of Coulomb interactions, their inertness, and their non-existent or merely low tendency for metal ions to complex.

The great advantage here is that the surface-active antioxidant itself forms a “protective casing” at the phase boundary of the micelles between the hydrophilic and lipophilic phase. This antioxidant protective casing thus surrounds and protects the lipophilic phase in the interior of the micelle structures and/or one or more (lipophilic) active substances contained therein. It provides a double function as an antioxidant and emulsifier. In this case, the emulsifying properties are comparable to those of known emulsifiers that are available for purchase, such that the surface-active antioxidant can degrade the surface tension sufficiently without a co-emulsifier that a microemulsion spontaneously develops when stirring.

By means of the combined action of a surface-active antioxidant (having a corresponding double function as an antioxidant and a surface-active agent) and the special properties of the zwitterionic substance, in particular the zwitterionic buffer, a good solution and a stable formulation of active substances that are at least sparingly soluble in water and are in particular sensitive in the form of a nanoemulsion/microemulsion formation also results as a suspension in a specific embodiment. In addition, this combination results in good biocompatibility and bioavailability.

Zwitterionic buffers are molecules that have an identical number of cationic and anionic functional groups. Particularly when using zwitterionic buffers instead of the standard organic or inorganic buffers, owing to its inertness and low interaction with other molecules, further stabilization e.g. against oxidative degradation and/or complexing of metal ions contained in the active substance can be observed. This contributes to the stabilization of e.g. a metal-ion-containing active substance in the solution, since the metal ions are not complexed, by contrast with many organic buffers, and therefore no sedimentation or phase separation can be observed.

The simplest example is amino acids, such as glycine, having an acid function and a base function. The carboxyl group loses a hydrogen ion and has a negative charge, and the amino group gains a hydrogen ion and has a positive charge. Other examples are the buffer substances HEPES, MES, HEPPS, and MOPS derived from 2-amino-ethanesulfonic acid and 3-amino-propanesulfonic acid. Here, the sulfonic acid forms the anionic functional group —SO₃⁻ and the protonated secondary or tertiary ammonium group forms the cationic functional group.

The more complicated nature of its electrodynamic charge transfers within the molecule results in a pH dependency of its state of charge and its dipole moments. The charge densities can accordingly be modulated in a pH-dependent manner. This results in very high polarity in the molecule, but the molecule itself is electrically neutral overall. These buffers accordingly do not contribute to the ionic strength of a formulation. Strong dipole moments cause electrostatic interactions, however. Compared with the salt-based buffers, the specific structure results in other properties that are advantageous for the formulation of sensitive and slightly soluble active substances.

This is particularly beneficial in interaction with surface-active antioxidants for the formulation of sensitive and/or slightly soluble active substances in microemulsions or nanoemulsions.

One or more lipophilic components are preferably located in the interior of the micelle structures made of surface-active antioxidants. The lipophilic component can either solely be the lipophilic active substance that is at least sparingly soluble in water and/or one or more lipophilic (carrier) components, in which the active substance is incorporated and additionally protected. The (lipophilic) active substance can also act solely as a nourishing substance or protective substance at the application site.

The weight ratio of the lipophilic component (without taking into account the at least one active substance that is at least sparingly soluble in water, as is present in this case) to the surface-active antioxidant is preferably between 1:4 and 1:10, more preferably between 1:1.45 and 1:1.9, and particularly preferably 1:1.5 and 1:1.8.

Especially hydrolysis-sensitive active substances that are at least sparingly soluble in water can also receive additional protection against hydrolytic and/or photolytic degradation by being embedded in a lipophilic carrier component. Photolytic degradation into the lipophilic phase can be enhanced by additional antioxidants such as carotenoids, Q 10, riboflavin, ascorbyl palmitate, and the like.

The protective effect is particularly effective owing to the double function of the surface-active antioxidant because:
the antioxidant is not freely movable in the emulsifier layer or even in the entire lipophilic phase of the micelle, but forms a unit with the emulsifier molecules and therefore a structure distributed completely evenly over the entire hydrophilic/lipophilic boundary surface, comparably to a protective film or an antioxidant protective barrier. Evenly equipping the phase boundary with antioxidants in this way is a key factor.
The concentration of the antioxidant is equal to that of the emulsifier (double function of a molecule).

A specific embodiment of the self-emulsifying oil-in-water microemulsion or nanoemulsion according to the invention provides that a first part of the at least one active substance that is at least sparingly soluble in water is present in micelles so as to be encapsulated by the at least one surface-active antioxidant and a second part is contained in the aqueous phase, for example in a dispersed and/or dissolved manner, wherein the weight ratio of the first part to the second part is preferably 1:10 to 10:1, preferably 2:10 to 10:2.

According to a preferred embodiment, the at least one active substance that is at least sparingly soluble in water is selected from the group consisting of pharmacologically active substances that are at least sparingly soluble in water or cosmetics that are at least sparingly soluble in water.

The pharmacologically active substances that are at least sparingly soluble in water are preferably selected from the group consisting of
antiseptic agents, such as bibrocathol, chlorhexidine,
anti-infective agents, preferably antibiotics, such as chloramphenicol, chlortetracycline, tetracycline, oxytetracycline, ofloxazin,
virucidal agents and virostatic agents, such as azelastine and azelastine hydrochloride, anti-inflammatory agents, preferably corticosteroids, such as cortisone, dexamethasone, prednisolone, fluorometholone, budesonide, betamethasone, fluticasone, fluticasone propionate, fluticasone furoate, mometasone, mometasone fuorate, or triamcinolone, and derivatives;
non-steroidal anti-inflammatory agents, such as diclofenac, nepafenac, bendazac, and derivatives, salicylic acid and derivatives, acetyl salicylic acid, paracetamol, ibuprofen, and mydriatic agents and cycloplegic agents, preferably anticholinergic agents such as atropine and atropine sulfate.

In particular, the at least one pharmacologically active substance is an antiseptically acting substance selected from the group consisting of 4,5,6,7-tetrabromo-1,3, A2-benzodioxabismol (bibrocathol), non-steroidal anti-inflammatory agents, in particular 2-amino-3-benzoylbenzeneacetamide (nepafenac), and mixtures and combinations thereof.

Exemplary cosmetics that are at least sparingly soluble in water are selected from the group consisting of argan oil, aloe vera oil, apricot kernel oil, arnica oil, avocado oil, calendula oil, marigold oil, peanut oil, St. John's wort oil, coconut oil, castor oil, and essential oils such as menthol, eucalyptus oil, thymol oil, lemon oil, orange oil, lemon oil, and the like.

In addition to the active substances that are at least sparingly soluble, the self-emulsifying oil-in-water microemulsion or nanoemulsion according to the invention can also contain water-soluble active substances which are dissolved in the aqueous phase. Examples of this are valaciclovir, gentamicin sulfate, kanamycin sulfate, levofloxacin, flunisolide, xylometazoline, xylometazoline hydrochloride, pseudoephedrine, and mixtures and combinations thereof.

A further preferred embodiment provides that the at least one surface-active antioxidant is selected from the group consisting of
vitamin derivatives, such as α-tocopheryl polyethylene glycol 1000 succinate (vitamin E TPGS, CAS-No.: 9002-96-4), ascorbyl palmitate, retinyl palmitate, tocopherol and tocotrienol derivatives, for example;
derivatives of alkyl gallates, in particular glycosylated alkyl gallates (C4-C18), such as epigallocatechin-3′-O-alphaglycoside
and mixtures and combinations thereof.

The molar content of the at least one zwitterionic substance is advantageously 1 to 100 mmol/l, preferably 2 to 50 mmol/l, more preferably 5 to 25 mmol/l, particularly preferably 5 to 20 mmol/l, in respect of the self-emulsifying oil-in-water microemulsion or nanoemulsion.

Alternatively or additionally, the weight ratio of the at least one zwitterionic substance is preferably from 0.02 to 3.0 wt. %, preferably from 0.10 to 0.60 wt. %, particularly preferably from 0.20 to 0.45 wt. %, in respect of the self-emulsifying oil-in-water microemulsion or nanoemulsion.

The at least one zwitterionic substance is preferably selected from the group consisting of
zwitterionic buffers, particularly preferably at least one zwitterionic buffer selected from the group consisting of Good's buffers, in particular a Good's buffer having a pKs value of between 6 and 8.5, such as 3-(N-morpholinopropane-1-sulfonic acid) (MOPS), 2-hydroxy-3-morpholinopropanesulfonic acid (MOPSO), 2-(4-(2-hydroxyethyl)-1-piperazine) ethanesulfonic acid (HEPES), (2,2′-(1,4-piperazine)diyldiethanesulfonic acid (PIPES), 2-{[1,3-dihydroxy-2-(hydroxymethyl)propan-2-yl]amino)ethane-1-sulfonic acid (TES), 2-[(2-amino-2-oxoethyl)-(carboxymethyl)amino] acetic acid (ADA), (N,N-bis(2-hydroxyethyl)-2-amino ethanesulfonic acid (BES), N-tris-(hydroxymethyl)methyl-2-amino-ethanesulfonic acid (TES), 2-hydroxy-3-[4-(2-hydroxyethyl)piperazin-1-yl]propane-1-sulfonic acid (HEPPSO), 4-(2-hydroxyethyl)-piperazine-1-propanesulfonic acid (HEPPS), (N-(2-hydroxyethyl)piperazine-N′-(4-butanesulfonic acid) (HEPBS), 3-N-bis-(hydroxyethyl)-amino-2-hydroxy-propanesulfonic acid (DIPSO), N-tris(hydroxymethyl)methyl-2-aminoethanesulfonic acid (TES), and mixtures and combinations thereof, such as MOPS/HEPES and MOPS/HEPES/DIPSO, amino acids, in particular carnitine, cysteine, L-leucine, L-lysine hydrochloride, L-proline, glycine, and derivatives thereof such as N-acetyl cysteine, arginine, ornithine, glutamine, aminoethanesulfonic acids, in particular taurine,
betaine,
and mixtures and combinations thereof.

The at least one zwitterionic substance is preferably selected from the group consisting of zwitterionic buffers and is preferably contained in molarities in the range of 1 to 50 mM, more preferably 2 to 25 mM, particularly preferably 5 to 20 mM, in the self-emulsifying oil-in-water microemulsion or nanoemulsion.

The zwitterionic substance is preferably selected from the wider group of Good's buffers, which no longer only trace back to Good et al. They are currently mainly used for biochemical purposes and cell cultures. In the following, some properties of Good's buffers are set out and it is explained why these buffers, in addition to the current use in simple biochemical tests such as electrophoresis and protein determination, are now particularly advantageously used in the present invention in the preparation and storage of a microemulsion or nanoemulsion.

When using Good's buffers, there were no changes to the formulations, such as precipitation or discoloration.

These are buffers having a pKs value of between 6 and 8.5 and at least one of the following properties
a. low solubility in non-polar components (such as lipids, oils); this property is advantageous for preventing buffer components from accumulating in the lipophilic phase of the micelles, and where necessary from interacting therewith or with active substances contained therein, and/or
b. a minimal influence of the temperature, ionic strength, or buffer concentration on the pKa value; this is advantageous for avoiding pH-dependent hydrolysis, and/or
c. optimally (but not necessarily), no or only low complexing properties for polyvalent metal ions, in particular Cu, Mg, Ni, Co; this is advantageous with metal-containing active substances such as bibrocathol.

These buffers are known for being inert and stable, for interacting less with other components, and for not passing through membranes. Therefore, they are advantageous as additional protective components in the emulsion according to the invention.

The Good's buffers can for example also be present in combination with TRIS, e.g. TES-TRIS (TEST), HEPES:TRIS (HEPEST), MOPS:TRIS (MOPST) and/or PIPES:TRIS (PIPEST).

In this case, the organic zwitterionic buffers used can be present both as free acids and so as to be conjugated with salts, e.g. as sodium salt conjugates.

The advantages of the zwitterionic buffers, in this case in particular also the Good's buffers, are known in conjunction with laboratory tests, e.g. electrophoretic measurements, protein determination, and protein renaturation, molecular-biological experiments, and also in cell culturing.

The microemulsion or nanoemulsion according to the invention is, however, preferably free of citrate buffer, since it can remove metal ions from metal-ion-containing active substances by complexing.

It is also advantageous that these buffers are inert in the tissue to which the formulation is applied, i.e. on the surface of the eye, for example. They do not undergo any enzymatic or non-enzymatic changes, i.e. they are not an enzyme substrate or enzyme inhibitor and do not react with metabolites or other components. Many of the currently standard buffers in eye drops do not meet these requirements.

It has surprisingly been found that these buffers can advantageously be used in the formulation of active substances that are at least sparingly soluble in water in microemulsions or nanoemulsions. In particular, formulations containing HEPES and/or MOPS have proven to be extremely stable, although they have not previously been used in eye drops.

In this case, a further preferred zwitterionic substance is vitamin E TPGS.

In particular in combination with vitamin E TPGS, improved stability, bioavailability and biocompatibility of the active substance that is at least sparingly soluble in water and of the formulation can be obtained. The zwitterionic buffers have the following advantages over mineral buffers:
pKa values closer to the physiological pH (between 6 and 8)
low penetration through biological membranes
concentration, temperature, and ion composition of the medium have only a minimal influence on the buffer capacity
any metal ions that are present are usually not complexed, or are only complexed to a limited extent

Owing to the above-described properties, the combination of zwitterionic buffers and surface-active antioxidants (preferably vitamin E TPGS) has a particularly advantageous effect on:
the “encapsulation” of easily degradable and/or slightly soluble active substances in surface-active micelles
the storage stability
the biocompatibility and compatibility of the formulation
the bioavailability

Zwitterionic buffers have very good water solubility and do not contribute to the ionic strength. They enhance the hydrophobic interactions, known e.g. from protein renaturation experiments.

It has been found that enhancing the hydrophobic interactions also encourages the micelle formation during the preparation process and increases the micelle stability. Accordingly, an active substance that is at least sparingly soluble in water is more rapidly and effectively encapsulated in the micelles than is the case when using mineral buffers. Furthermore, the exposure of the active substance that is at least sparingly soluble in water to the buffer solution during the preparation process, caused by enhancing the hydrophobic interactions due to the more rapid micelle formation, is minimized. This is an important aspect with hydrolysis-sensitive active substances, for example. The simultaneous use of a surface-active antioxidant as a solubilizer protects the active substance that is at least sparingly soluble in water against degradation processes which can be initiated by reactive oxygen species, for example. These can develop during preparation due to UV light or by being catalysed by iron ions, which are often present in chemicals as trace impurities.

When metal-ion-containing active substances that are at least sparingly soluble in water, such as bibrocathol, are used, the low metal-ion complexing properties of zwitterionic buffers are likewise advantageous. For instance, the metal-containing active substance is protected against degradation by complexing of the metal ions during the encapsulation process, i.e. the micelle formation process, and also during storage of the formulation. The latter is brought about by zwitterionic buffers not permeating through membranes.

The formulation according to the invention also provides improved biocompatibility after application to the site of action. This is e.g. likewise brought about by the low metal-ion complexing properties of zwitterionic buffers. As a result, inactivation of enzymes at the site of action and/or formation of slightly soluble precipitates (calcium deposit on the cornea) can be prevented, for example.

By contrast therewith, the mineral buffers have the various corresponding drawbacks.

Improved bioavailability can be brought about by electrostatic interaction of the zwitterionic buffers with membranes, which can make it easier to take up active substances. At the same time, vitamin E TPGS, isopropyl myristate, and/or isopropyl palmitate also act as permeation enhancers, the use of which is described as being preferred in the application.

Interaction of zwitterionic buffers having their special properties and surface-active antioxidants having a corresponding double function results in excellent protection and stabilization of nanoemulsions and microemulsions and improved bioavailability.

Particularly preferably, the self-emulsifying oil-in-water microemulsion or nanoemulsion according to the invention is free of mineral buffers, in particular phosphate buffers and borate buffers, tromethamol (TRIS), polysorbate 80, Cremophor EL, cyclodextrins, medium-chain triglycerides, and/or omega-3 fatty acids.

The total content of the at least one active substance that is at least sparingly soluble in water is preferably 0.001 to 5.0 wt. %, preferably 0.01 to 1.0 wt. %, particularly preferably 0.02 to 0.10 wt. %, in particular 0.04 to 0.08 wt. %, or 0.1 to 0.5 wt. %, in respect of the oil-in-water microemulsion or nanoemulsion.

It is likewise advantageous for the total content of the at least one surface-active antioxidant to be in quantities of 0.01 to 10 wt. %, preferably 0.1 to 5.0 wt. %, particularly preferably 0.5 to 2.0 wt. %, in particular 1.4 to 1.7 wt. %, in respect of the self-emulsifying oil-in-water microemulsion or nanoemulsion.

The combination according to the invention of surface-active antioxidant and zwitterionic substance allows for an excellent solution and bioavailability of the active substance that is at least sparingly soluble in water, such that this can take effect even when low concentrations are used. The active substance that is at least sparingly soluble in water can therefore be used in low concentrations, meaning that the compatibility of the composition according to the invention is increased.

The self-emulsifying oil-in-water microemulsion or nanoemulsion according to the present invention can contain at least one further additive (or excipient) which is preferably selected from the group consisting of
a) antioxidants, in particular carotenoids, such as alpha, beta, gamma carotine, capsanthin, lycopene, zeaxanthin, astaxanthin, luteine, coenzyme Q, EDTA, bile acid, and their derivatives, e.g. alkyl gallates (C4-C18), and derivatives of the alkyl gallates, in particular derivatives of propyl gallate, octyl gallate, and dodecyl gallate, lecithin, butylhydroxytoluene (BHT), butylhydroxyanisole (BHA), and mixtures and combinations thereof, which antioxidants differ from the at least one surface-active antioxidant,
b) lipophilic components, in particular isopropyl myristate, isopropyl palmitate, Miglyol, paraffins, medium-chain triglycerides, algae oil, fish oil, jojoba oil, sea buckthorn oil, sesame oil, isopropyl myristate (IMP) and mixtures and combinations thereof, c) isotonizing agents, in particular NaCl and/or alditols, such as sorbitol, mannitol, glycerol, and the like,
d) viscosity improvers such as xanthan gum, glycosaminoglycanes, such as hyaluronic acid or salts thereof, chondroitin sulfate, cellulose derivatives, in particular hydroxypropylmethylcellulose (HPMC),
e) humectants and lubricants such as glycerol, polyethylene glycols,
and mixtures and combinations thereof.

It was possible to observe that the presence of excipients, in particular of viscosity improvers, improves the stability of the emulsion. The addition of glycerol and PEG 400 also results in visually clearer formulations.

Substances that are preferably contained such as glycerol, polyethylene glycols, and the like act as humectants and lubricants which improve the compatibility with the eye in suspensions, for example.

Preferred lipophilic carrier substances are e.g. isopropyl myristate, oleic acid, and Miglyol.

A particularly preferred formulation contains e.g. a lipophilic matrix of 0.2-0.3% for the lipophilic carrier substance, e.g. isopropyl myristate, and 1.4-1.7% for the emulsifier vitamin E TPGS, as found to be optimal for the uptake and stabilization of the tested bibrocathol concentrations, insofar as this can be determined by visual inspection. This corresponds to a carrier oil to vitamin E TPGS ratio of 1:4.6 to 1:8.5.

If lipophilic components are contained in the self-emulsifying oil-in-water microemulsion or nanoemulsion, the total weight ratio of the lipophilic components (without taking into account the lipophilic active substance that may be present) to vitamin E TPGS is between 1:4 and 1:10, preferably 1:1.45:1:1.9, and particularly preferably 1:1.5 to 1:1.8.

According to a particularly preferred embodiment, the self-emulsifying oil-in-water microemulsion or nanoemulsion contains
vitamin E TPGS as a surface-active antioxidant, preferably in a quantity of 0.01 to 10 wt. %, preferably 0.1 to 5.0 wt. %, particularly preferably 0.5 to 2.0 wt. %, in particular 1.4 to 1.7 wt. %,
HEPES and/or MOPS as a zwitterionic substance, preferably in a total quantity of 1 to 100 mmol/I, preferably 2 to 50 mmol/l, particularly preferably 5 to 20 mmol/I,
bibrocathol as a pharmacologically active substance, preferably in a quantity of 0.001 to 5.0 wt. %, preferably 0.01 to 1.0 wt. %, particularly preferably 0.1 to 0.5 wt. %, in each case in respect of the self-emulsifying oil-in-water microemulsion or nanoemulsion, and up to 100 wt. % water.

In this embodiment, it is preferable for the self-emulsifying oil-in-water microemulsion or nanoemulsion according to the invention to additionally contain
Q10, BHT, and/or propyl gallate as antioxidants which differ from the at least one surface-active antioxidant, preferably in a total quantity of 0.01 to 1.0 wt. %, preferably 0.02 to 0.5 wt. %, particularly preferably 0.04 to 0.25 wt. %,
xanthan gum as a viscosity improver, preferably in a quantity of 0.01 to 2.0 wt. %, preferably 0.05 to 1.0 wt. %, particularly preferably 0.1 to 0.4 wt. %, and/or
IMP and/or Miglyol as lipophilic components, preferably in a quantity of 0.01 to 2.0 wt. %, preferably 0.05 to 1.0 wt. %, particularly preferably 0.1 to 0.4 wt. %,
   in each case in respect of the self-emulsifying oil-in-water microemulsion or nanoemulsion.

The self-emulsifying oil-in-water microemulsion or nanoemulsion can contain preservatives known from the prior art. The self-emulsifying oil-in-water microemulsion or nanoemulsion is preferably free of preservatives, however.

The self-emulsifying oil-in-water microemulsion or nanoemulsion according to any one of the preceding claims is suitable as a pharmaceutical product, which is suitable in particular for topical application, preferably for use as an ophthalmic agent, for nasal application, and/or for application in the oral or throat area, in particular in the form of eye drops, nasal drops, and/or for use as an antiseptic agent, for example for use in a method for the prophylaxis and treatment of infections and allergies in the region of the eyes, nose, and/or dry eyes (keratoconjunctivitis sicca, sicca syndrome) or in the oral/throat area as a spray or rinse for the prophylaxis or treatment of respiratory tract infections, colds, sore throats, or inflammation.

Alternatively, the self-emulsifying oil-in-water microemulsion or nanoemulsion can be used as a cosmetic which is in particular for application in the oral or throat area, in particular in the form of a spray or rinse, for example to help with easier breathing and fresh breath.

According to another embodiment, the present invention relates to an emulsifying composition, containing or consisting of
at least one surface-active antioxidant, and
at least one zwitterionic substance, wherein
the composition is free of active substances that are at least sparingly soluble in water, in particular pharmacologically active substances that are at least sparingly soluble in water and/or cosmetics that are at least sparingly soluble in water.

The emulsifying composition is in particular in the form of an aqueous solution.

The total content of the at least one surface-active antioxidant is preferably 0.1 to 5.0 wt. %, preferably 0.5 to 2 wt. %, in particular 1.4 to 1.7 wt. %, in respect of the emulsifying composition.

For the emulsifying composition, it is likewise preferable for the total content of the at least one zwitterionic substance to be in quantities of 1 to 100 mmol/l, preferably 2 to 50 mmol/l, more preferably 5 to 25 mmol/l, particularly preferably 5 to 20 mmol/l, in respect of the emulsifying composition.

The present invention will be explained in greater detail with reference to the following configurations without restricting the invention to the embodiments set out.

By means of the formulation according to the invention, it is possible, for example, to process the metal-containing and slightly soluble active substance bibrocathol, which has previously only been formulated as an ointment, in the form of aqueous eye drops. (Bibrocathol ranks among antiseptic agents and is used for the prophylaxis and treatment of infections in the region of the eyes.)

In this case, bismuth-containing, slightly soluble bibrocathol is packaged in micelles formed by the surface-active antioxidant vitamin E TPGS and is thus effectively protected against contact with water and degrading influences (such as oxygen radicals, UV rays, complexing agents, and the like). Additional components of the formulation, such as the selected zwitterionic buffer, increase the protection.

Corresponding O/W microemulsions or nanoemulsions according to the invention also make it possible to effectively protect other slightly soluble and/or sensitive and/or easily degradable substances, e.g. hydrolysis-sensitive, oxidation-sensitive, photolabile substances in micelle structures, formed by antioxidants, against degradation.

The self-emulsifying O/W microemulsions or nanoemulsions according to the invention are in particular based on:
1) a surface-active antioxidant, e.g. vitamin derivatives, such as α-tocopheryl polyethylene glycol 1000 succinate (vitamin E TPGS), ascorbyl palmitate, derivatives of alkyl gallates and
2) a zwitterion, e.g. zwitterionic buffer, preferably selected from the extended group of Good's buffers
with the aim of effectively protecting slightly soluble and/or sensitive and/or easily degradable substances, e.g. hydrolysis-sensitive, oxidation-sensitive, photolabile substances in micelle structures, formed by antioxidants, against degradation.

The present invention will be described in greater detail with reference to the following configurations without restricting the invention to the embodiments set out.

### EXAMPLES 1-9 ACCORDING TO THE INVENTION

### Lipophilic Phase

The active substance that is at least sparingly soluble in water and the lipophilic carrier components (e.g. IMP) are mixed at 37-42° C., then the surface-active antioxidant (e.g. vitamin A TPGS) is added and is stirred at 37° C. until all the components are homogeneously emulsified.

### Mixture of the Hydrophilic and Lipophilic Phase

The above-mentioned lipophilic phase is then mixed with the zwitterionic buffer pre-heated to 37-42° C. and is stirred well at 250 to 500 rpm until a homogeneous nanoemulsion or microemulsion is formed.

The further hydrophilic components are then successively added and are stirred each time until all the substances added in this step are finally dissolved. The viscosity improver (e.g. xanthan gum) is added as the last component here.

The examples set out in the following table have been prepared using the approach above.

### EXAMPLES

Formulation composition

| | 1 | 2 | 3 | 4 | 5 | 6 | 7 | 8 | 9 |
|---|---|---|---|---|---|---|---|---|---|
| Composition | [%] wt/wt | [%] wt/wt | [%] wt/wt | [%] wt/wt | [%] wt/wt | [%] wt/wt | [%] wt/wt | [%] wt/wt | [%] wt/wt |
| Bibrocathol | 0.04 | 0.04 | 0.04 | 0.05 | 0.06 | 0.08 | 0.1 | 0.1 | 0.1 |
| IMP | 0.2 | | 0.25 | 0.2 | 0.2 | 0.2 | 0.2 | 0.2 | 0.2 |
| Miglyol | | 0.3 | | | | | | | |
| Vitamin E TPGS | 1.4 | 1.4 | 1.4 | 1.46 | 1.5 | 1.5 | 1.6 | 1.6 | 1.4 |
| Q10 | | | | 0.01 | | | | | |
| BHT | 0.06 | 0.08 | 0.2 | | | | | | 0.06 |
| Propyl gallate | | | | | 0.05 | 0.05 | 0.05 | 0.05 | |
| NaCl | 0.2 | 0.05 | | | 0.2 | | | 0.3 | 0.2 |
| Sorbitol | | | | | | 0.3 | | | |
| Xanthan gum | 0.2 | 0.2 | 0.2 | 0.2 | 0.2 | 0.25 | 0.3 | 0.3 | 0.2 |
| HEPES | up to 100 | up to 100 | up to 100 | | | | | | |
| MOPS | | | | up to 100 | up to 100 | up to 100 | up to 100 | up to 100 | up to 100 |

It has surprisingly been found that the above-described formulations are stable and therefore do not result in any sedimentation or phase separation. Likewise, the pharmaceutically active constituent (bibrocathol) is effectively protected against oxidation owing to the effective encapsulation with an antioxidant.

### EXAMPLE 10

The present invention is also explained on the basis of the following examples, in which a stable formulation of bibrocathol that is protected against oxidation has been formulated as an O/W microemulsion, stabilized by a surface-active antioxidant and a zwitterionic buffer which is suitable for use as eye drops.

D-α-tocopheryl polyethylene glycol 1000 succinate (vitamin E TPGS) was selected as the ophthalmologically acceptable surface-active antioxidant. Of the many that were tested, isopropyl myristate, oleic acid, and Miglyol were found to be suitable as lipophilic carrier substances.

The zwitterionic buffers MOPS and HEPES were extremely suitable for stabilizing the emulsion, not only in respect of the pH value.

In this case, the concentration ranges of 0.15 to 0.3 wt. % for the lipophilic carrier substances and 1.4-1.7 wt. % for vitamin E TPGS have been found to be optimal for the uptake and stabilization of the tested bibrocathol concentrations of 0.04-0.08%.

This is evaluated by a purely visual assessment of the stability of the microemulsion.

The antibacterial efficacy of the formulations against Staphylococcus aureus was demonstrated in the zone of inhibition test. Although only low quantities of bibrocathol were taken up in the formulation, an inhibition effect could be demonstrated. This allows a conclusion to be drawn on the action against an application-based microorganism.

Tests show that one part of the bibrocathol is encapsulated in the micelles and one part is free in the aqueous phase. Bibrocathol that is free in the solution is rapidly available bibrocathol, and bibrocathol that is contained in the micelles is released with a delay (retard effect). Viscosity improvers, in particular muco-adhesive viscosity improvers, can also encourage this effect.

### EXAMPLE 11

### Production of the Microemulsion

Bibrocathol was stirred in a lipophilic matrix consisting of a carrier substance, the surface-active antioxidant vitamin E TPGS, and further lipophilic excipients according to the composition set out in the following table under heating to approx. 42° C. until homogeneity was reached.

| | |
|---|---|
| | Concentration |
| Components | [wt. %] |

Lipophilic matrix

| | |
|---|---|
| Bibrocathol | 0.04 |
| Isopropyl myristate | 0.3 |
| Vitamin E TPGS | 1.6 |
| BHT | 0.04 |

Aqueous phase

| | |
|---|---|
| Glycerol | 1.8 |
| HPMC | 0.4 |
| HEPES buffer (20 mM) | Up to 100 |

BHT: butylhydroxytoluene . . .
HPMC: hydroxypropylmethylcellulose
HEPES: 2-(4-(2-hydroxyethyl)-1-piperazine) ethanesulfonic acid

Mixing the lipophilic matrix with the aqueous phase, likewise pre-heated to 42° C., results in a stable, self-emulsifying, opaque to clear formulation.

### EXAMPLES 12-15: ZONE OF INHIBITION TEST

To carry out the zone of inhibition test, Staphylococcus aureus-containing material was smoothed onto a solid growth medium. Small filter paper disks, which were each impregnated with a particular eye drop solution, were placed onto the applied bacteria layer. The formulation diffused into the solid growth medium. If the formulation contained active substance, the bacterial growth was inhibited and clearly visible zones of inhibition developed. A zone of inhibition is the clear region between the edge of the filter plate and the start of a cell colony. If a filter paper disk did not exhibit a zone of inhibition, either not enough active substance diffused into the solid growth medium or no more active substance was available (degradation).

In this case, the zone of inhibition test makes it possible to directly compare the efficacy of the different bibrocathol-containing formulations tested. Here, the influence of formulation constituents and concentrations on the availability of the antibacterial active substances could also be tested and compared. The size of the zone of inhibition is a measure of the availability and efficacy of the inhibition substance here.

The following formulations were tested; here, 0.04 wt. % bibrocathol was taken up into different lipophilic carrier substances in each case (stated in wt. %):

| | | Carrier | Vitamin | Propyl | | | | Xanthan | |
|---|---|---|---|---|---|---|---|---|---|
| Example | Bibrocathol | substance | E TPGS | gallate | BHT | NaCl | | gum | Buffer |
| 12 | 0.040 | IMP | 1.4 | | 0.08 | 0.05 | | 0.02 | HEPES |
| | | 0.250 | | | | | | | up to |
| | | | | | | | | | 100 |
| 13 | 0.040 | Miglyol | 1.4 | | 0.08 | 0.05 | | 0.20 | HEPES |
| | | 0.250 | | | | | | | up to |
| | | | | | | | | | 100 |
| 14 | 0.040 | Oleic acid | 0.68 | 0.05 | | 0.2 | Kolliphor | 0.20 | MOPS |
| | | 0.240 | | | | | P188 | | up to |
| | | | | | | | 0.22 | | 100 |
| 15 | 0.040 | Paraffin Per | 1.6 | | 0.05 | | HPMC | 0.15 | HEPES |
| | | 0.280 | | | | | 0.28 | | up to |
| | | | | | | | | | 100 |

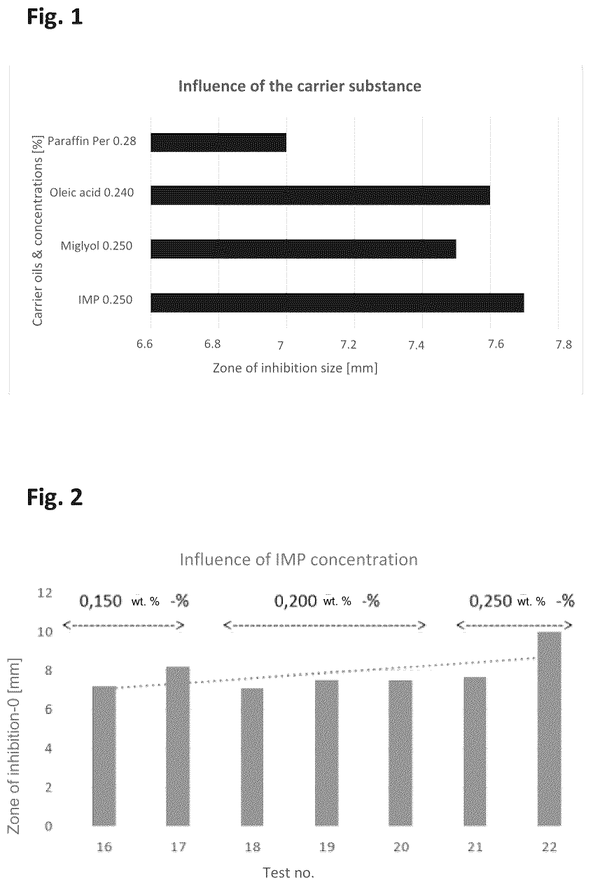

The results for the zone of inhibition size are shown in FIG. 1.

All the tests show positive results in the zone of inhibition test. The formulation containing the carrier substance isopropyl myristate brought about the largest zone of inhibition, followed by oleic acid and Miglyol. The paraffin per-containing formulation brought about the smallest zone of inhibition by far.

### EXAMPLES 16-22

In the formulations in examples 16-22, the influence of the concentration of isopropyl myristate on the zone of inhibition size was tested.

The following formulations were tested here (all stated in wt. %):

| | | | Vitamin | Propyl | | | Xanthan | |
|---|---|---|---|---|---|---|---|---|
| Example | Bibrocathol | IMP | E TPGS | gallate | BHT | NaCl | gum | Buffer |
| 16 | 0.04 | 0.150 | 1.4 | | 0.02 | 0.05 | 0.2 | HEPES |
| 17 | 0.04 | 0.150 | 1.4 | | 0.08 | 0.05 | 0.2 | HEPES |
| 18 | 0.04 | 0.200 | 1.4 | | 0.06 | 0.2 | 0.2 | HEPES |
| 19 | 0.04 | 0.200 | 1.4 | 0.05 | | | 0.2 | HEPES |
| 20 | 0.04 | 0.200 | 1.4 | 0.05 | | 0.2 | 0.2 | MOPS |
| 21 | 0.04 | 0.250 | 1.4 | | 0.08 | 0.05 | 0.2 | HEPES |
| 22 | 0.04 | 0.250 | 1.4 | | 0.08 | — | 0.2 | HEPES |

Again, all the formulations gave a positive result in the zone of inhibition test. An increasing concentration of the lipophilic carrier substance isopropyl myristate with a constant bibrocathol concentration of 0.04 wt. % in the formulations results in a slight trend toward larger zones of inhibition, as shown by the trend line in FIG. 2. This effect can be attributed to a slightly higher solubility of bibrocathol or further diffusion in the growth medium on the test plates due to higher isopropyl myristate concentrations.

### EXAMPLES 23-35

Likewise, the influence of the concentration of the bibrocathol on the zone of inhibition size was tested on the basis of the following formulations (all stated in wt. %):

| | | ISOPROPYL | Vitamin | Propyl | | | | Xanthan |
|---|---|---|---|---|---|---|---|---|
| | Bibrocathol | MYRISTATE | E TPGS | gallate | Q10 | Sorbitol | NaCl | gum |
| Controls | | | | | | | | |
| C1 | — | 0.200 | 1.5 | 0.05 | | | | 0.25 |
| C2 | — | 0.200 | 1.5 | 0.05 | | | | 0.25 |
| Examples | | | | | | | | |
| 23 | 0.04 | 0.200 | 1.4 | 0.05 | | | 0.2 | 0.2 |
| 24 | 0.04 | 0.200 | 1.4 | 0.05 | | | | 0.2 |
| 25 | 0.06 | 0.200 | 1.5 | 0.05 | | 2.4 | 0.2 | 0.2 |
| 26 | 0.06 | 0.200 | 1.5 | 0.05 | | | 0.2 | 0.2 |
| 27 | 0.08 | 0.200 | 1.5 | | 0.04 | | | 0.2 |
| 28 | 0.08 | 0.200 | 1.5 | | 0.04 | | | 0.25 |
| 29 | 0.08 | 0.200 | 1.5 | 0.05 | | 0.3 | | 0.25 |
| 30 | 0.08 | 0.200 | 1.5 | 0.05 | | | | 0.25 |
| 31 | 0.080 | 0.200 | 1.5 | | 0.04 | | | 0.2 |
| 32 | 0.100 | 0.200 | 1.5 | 0.05 | | | 0.2 | 0.25 |
| 33 | 0.100 | 0.200 | 1.6 | 0.05 | | | | 0.3 |
| 34 | 0.100 | 0.200 | 1.5 | 0.05 | | | 0.1 | 0.2 |
| 35 | 0.100 | 0.200 | 1.6 | 0.05 | | | 0.3 | 0.2 |

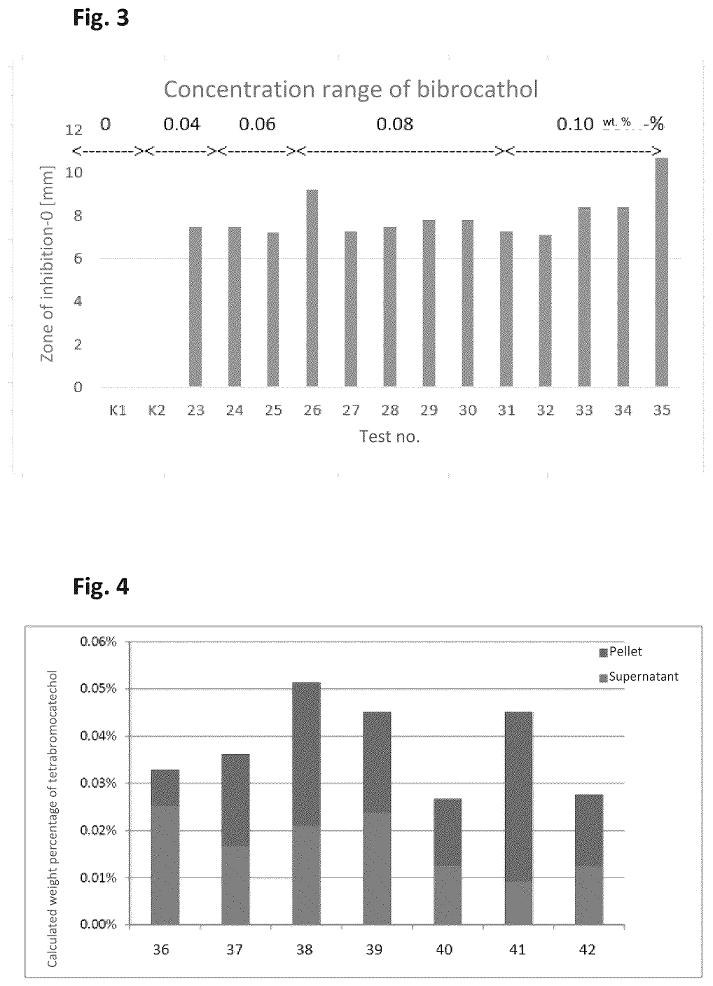

The results of this test series are reproduced in FIG. 3.

Again, all the tests according to the invention exhibited a positive zone of inhibition test. An increase in the bibrocathol concentration with a constant concentration of the lipophilic carrier isopropyl myristate in the formulations barely has any influence on the zone of inhibition size.

There are two possible explanations for this:
the solubility of bibrocathol in a constant concentration of lipophilic carrier substance isopropyl myristate is already achieved at the lowest concentration;
the diffusion in the growth medium on the test plates could be a limiting factor and could be promoted by the carrier substance.

Therefore, the formulations according to the invention exhibit excellent availability of the bibrocathol even at low concentrations, which indicates high bioavailability.

By comparison therewith, a control formulation having 0.1 wt. % bibrocathol in paraffin does not exhibit any zone of inhibition activity, even though sometimes considerably more bibrocathol is contained. The active substance diffusion and availability from the viscous paraffin is presumably considerably slower than in the aqueous microemulsion formulation.

### EXAMPLES 36-42

In order to measure the distribution of bibrocathol in the supernatant and the micelles, the aim was to separate the micelles from the formulation before the analysis, to transfer tetrabromocatechol (TBBC) into the degradation product, and to then detect this in both fractions. An analysis in a two-step process, i.e.:
1) separating the micelles from the aqueous formulation
2) analyzing the degradation product (TBBC) in both fractions by means of HPLC-MS.

A suitable separation method is ultracentrifugation of the formulations using Nanosep centrifugal filters from Pall having an appropriately selected pore size. The individual batches were ultracentrifuged at 42,000 rpm and 25° C. for 24 hours over the centrifugal filters (in accordance with AAPS PharmSciTech, Vol 10, No. 4, Dec. 2009).

The supernatant was then separated from the pellet and TBBC was determined in both fractions.

The following formulations were tested (all stated in wt. %):

| | | Oleic | | | Kolliphor | Vitamin E | MOPS 10 |
|---|---|---|---|---|---|---|---|
| Example | Bibrocathol | acid | Vit E | PEG 400 | P 188 | TPGS | mM |
| 36 | 0.040 | | | 0.4 | | 1.5 | up to 100 |
| 37 | 0.040 | | 0.1 | | | 0.8 | up to 100 |
| 38 | 0.050 | 0.5 | | | — | 1.5 | up to 100 |
| 39 | 0.040 | 0.5 | | | — | 1.5 | up to 100 |
| 40 | 0.025 | 0.5 | | | — | 1.5 | up to 100 |
| 41 | 0.050 | 0.5 | | | 0.47 | 1.5 | up to 100 |
| 42 | 0.025 | 0.5 | | | 0.47 | 1.5 | up to 100 |

The results shown in FIG. 4 demonstrate that one part of the TBBC is found in the supernatant and one part in the pellet. These results suggest that, during the preparation of the microemulsions, only one part of the bibrocathol is encapsulated in micelles and one part is present in the solution, optionally as a finely dispersed suspension solution.

From this distribution, an immediate effect and a retard effect can be assumed, in which directly active bibrocathol in the solution and bibrocathol that is released more slowly from the micelles take effect successively and in addition to one another.

1-24. (canceled)

25. A self-emulsifying oil-in-water microemulsion or nanoemulsion, comprising:
at least one surface-active antioxidant,
at least one zwitterionic substance, and
at least one active substance that is at least sparingly soluble in water, wherein
the at least one active substance that is at least sparingly soluble in water is present in micelles so as to be encapsulated at least in part by the at least one surface-active antioxidant.

26. The self-emulsifying oil-in-water microemulsion or nanoemulsion according to claim 25, wherein the at least one active substance that is at least sparingly soluble in water is selected from the group consisting of pharmacologically active substances or cosmetics that are at least sparingly soluble in water.

27. The self-emulsifying oil-in-water microemulsion or nanoemulsion according to claim 26, wherein a first part of the at least one active substance that is at least sparingly soluble in water is present in micelles so as to be encapsulated by the at least one surface-active antioxidant and a second part is contained in the aqueous phase.

28. The self-emulsifying oil-in-water microemulsion or nanoemulsion according to claim 26, wherein the pharmacologically active substances that are at least sparingly soluble in water are selected from the group consisting of antiseptic agents,
anti-infective agents,
virucidal agents,
virostatic agents,
anti-inflammatory agents,
non-steroidal anti-inflammatory agents, and
mydriatic agents and cycloplegic agents, and
combinations thereof.

29. The self-emulsifying oil-in-water microemulsion or nanoemulsion according to claim 26, wherein the pharmacologically active substance that is at least sparingly soluble in water is bibrocathol.

30. The self-emulsifying oil-in-water microemulsion or nanoemulsion according to claim 26, wherein the cosmetics that are at least sparingly soluble in water are selected from the group consisting of argan oil, aloe vera oil, apricot kernel oil, arnica oil, avocado oil, calendula oil, marigold oil, peanut oil, St. John's wort oil, coconut oil, castor oil, and essential oils such as menthol, eucalyptus oil, thymol oil, lemon oil, orange oil, and lemon oil.

31. The self-emulsifying oil-in-water microemulsion or nanoemulsion according to claim 25, wherein the at least one surface-active antioxidant is selected from the group consisting of α-tocopheryl polyethylene glycol 1000 succinate, ascorbyl palmitate, retinyl palmitate, and glycosylated alkyl gallates (C₄-C₁₈).

32. The self-emulsifying oil-in-water microemulsion or nanoemulsion according to claim 25, wherein
the molar content of the at least one zwitterionic substance is 1 to 100 mmol/l, and/or
the weight ratio of the at least one zwitterionic substance is from 0.02 to 3.0 wt. %,
in respect of the self-emulsifying oil-in-water microemulsion or nanoemulsion.

33. The self-emulsifying oil-in-water microemulsion or nanoemulsion according to claim 25, wherein the at least one zwitterionic substance is selected from the group consisting of zwitterionic buffers, amino acids, aminoethanesulfonic acids, and betaine.

34. The self-emulsifying oil-in-water microemulsion or nanoemulsion according to claim 33, wherein the at least one zwitterionic buffer is selected from the group consisting of Good's buffers.

35. The self-emulsifying oil-in-water microemulsion or nanoemulsion according to claim 33, wherein the zwitterionic buffer is contained in the range of 1 to 50 mM.

36. The self-emulsifying oil-in-water microemulsion or nanoemulsion according to claim 25, which is free of mineral buffers.

37. The self-emulsifying oil-in-water microemulsion or nanoemulsion according to claim 25, wherein a total content of the at least one active substance that is at least sparingly soluble in water is 0.001 to 5.0 wt. %, in respect of the oil-in-water microemulsion or nanoemulsion.

38. The self-emulsifying oil-in-water microemulsion or nanoemulsion according to claim 27, wherein a total content of the at least one surface-active antioxidant is in quantities of 0.01 to 10 wt. %, in respect of the self-emulsifying oil-in-water microemulsion or nanoemulsion.

39. The self-emulsifying oil-in-water microemulsion or nanoemulsion according to claim 25, wherein it contains at least one further additive selected from the group consisting of antioxidants, lipophilic components, isotonizing agents, viscosity improvers, humectants, and lubricants.

40. The self-emulsifying oil-in-water microemulsion or nanoemulsion according to claim 25, containing
vitamin E TPGS as a surface-active antioxidant,
HEPES and/or MOPS as a zwitterionic substance,
bibrocathol as a pharmacologically active substance that is at least sparingly soluble in water,
in each case in respect of the self-emulsifying oil-in-water microemulsion or nanoemulsion, and up to 100 wt. % water.

41. The self-emulsifying oil-in-water microemulsion or nanoemulsion according to claim 38, additionally containing
Q10, BHT, BHA, and/or propyl gallate as antioxidants which differ from the at least one surface-active antioxidant,
xanthan gum as a viscosity improver, and/or
IMP, IPP, and/or Miglyol as lipophilic components,
in each case in respect of the self-emulsifying oil-in-water microemulsion or nanoemulsion.

42. A pharmaceutical product comprising the self-emulsifying oil-in-water microemulsion or nanoemulsion according to claim 25.

43. The pharmaceutical product according to claim 42, which is a topical formulation, an ophthalmic formulation, a nasal formulation, an oral formulation, or a throat formulation.

44. A method of providing prophylaxis and treatment of infections and/or allergies in the region of the eyes, nose, and/or dry eyes, in a patient, the method comprising administering the pharmaceutical product of claim 42.

45. A method of providing costmetic application to a person comprising applying on the person a self-emulsifying oil-in-water microemulsion or nanoemulsion according to claim 25.

46. An emulsifying composition, containing
at least one surface-active antioxidant, and
at least one zwitterionic substance, wherein
the composition is free of active substances that are at least sparingly soluble in water.

47. The emulsifying composition according to claim 46, which is in the form of an aqueous solution.

48. The emulsifying composition according to claim 46, wherein a total content of the at least one surface-active antioxidant is 0.1 to 5.0 wt. % of the emulsifying composition.

49. The emulsifying composition according to claim 46, wherein the total content of the at least one zwitterionic substance is 1 to 100 mmol/l of the emulsifying composition.
